(19) United States
(12) Patent Application Publication
(10) Pub. No.: US 2016/0166323 A1
(43) Pub. Date: Jun. 16, 2016

(54) Sterile Drape for Hip Fracture Surgery

(71) Applicant: Angela Tylka, Chicago, IL (US)

(72) Inventor: Angela Tylka, Chicago, IL (US)

(21) Appl. No.: 14/569,659

(22) Filed: Dec. 13, 2014

(51) Int. Cl. A61B 19/10

(52) U.S. Cl. CPC A61B 19/10; A61B 2019/106

(57) ABSTRACT

A sterile isolation drape that can be used to create a vertical boundary to the sterile field during hip fracture and similar surgeries. The invention includes a cutout region or opening in the drape that interfaces with an elongated, closed plastic tunnel. This closed tunnel can receive the lower part of the C-arm and allow it to extend into position with respect to the patient without entering the sterile field. A fluid reservoir can be located laterally on one or both sides of the tunnel region to allow trapping of body fluids such as blood without interfering with the entry of the C-arm. The drape can be weighted on the bottom so that the vertical portion remains substantially vertical during entry and exit of the C-arm. The weights can be fixed or removable. The drape can also have optional utility pouches in convenient locations.

### FIELD OF THE INVENTION

The present invention relates generally to sterile drapes used in surgery and more particular to an improved sterile drape for hip fracture surgery that maintains the sterile field during use of a C-Arm radiography unit.

### DESCRIPTION OF THE PRIOR ART

During surgery, major parts of the operating suite are maintained in a sterile condition. This is accomplished by gowning the surgical team and by draping most non-sterile surfaces such as tables and the like with sterile drapes, including parts of the patient's body not undergoing surgery. Each sterile drape, when deployed, has a sterile top or outside and a non-sterile bottom or inside. Typically, anything the surgeon or members of the surgical team will touch during the procedure is maintained in a sterile condition using drapes or other devices such as sterilized light handles. Even after draping however, there are areas in the room that are considered non-sterile. The floor is a good example. The Association of Perioperative Registered Nurses, as well as other organizations, publish perioperative standards and recommended procedures. These standards define what is called the “sterile field”. The lower boundary of the sterile field is typically a horizontal plane passing through the patient's waist or across the top of the operating table. Anything below this plane is considered to be outside the sterile field, and hence non-sterile, anything above this plane up to an upper boundary is in the sterile field.

In some procedures such as hip fracture surgery, it is common practice to also define vertical boundaries to the sterile field. The region on the patient side of the vertical boundary above the horizontal sterile field boundary is in the sterile field, while the region on the opposite side of the vertical boundary is outside the sterile field. Clear plastic drapes called isolation drapes are typically used to create vertical boundaries. Large equipment such as 3-dimensional radiography equipment usually remains outside the sterile vertical boundary. A typical prior art plastic isolation drape is the Steri-Drape™ 6617 supplied by 3M Corporation of St. Paul, Minn. This sterile drape is supplied as a rectangle around 328 cm wide by 254 cm high (see FIG. 1). It has paper-covered sticky tape on the edges for hanging and is equipped with a central fluid pouch that captures body fluids such as blood. It also has an incise film located above the fluid pouch that can be coated with an antimicrobial such as that sold under the name of Ioban™.

During hip fracture and other orthopedic procedures, a 3-dimensional imaging radiography unit is used. The unit includes an x-ray source and detector mounted in opposing positions on a large C-arm. This allows the source and detector to be simultaneously rotated to any angle simply by rotating the C-arm. The C-arm can also be tilted. A typical C-arm radiography unit is manufactured by Siemens GmbH. Rotating and tilting the C-arm to different positions around the surgical site presents 3-dimensional images to the surgeon. These images are very important to assure the correct positioning of inserted screws and the like into bones. For example, a typical hip fracture procedure requires insertion of one or more lag screws into the femur. These screws must be positioned correctly in all directions.

The use of a C-arm poses problems with the sterile field. At almost any angle, part of the C-arm is below the level of the horizontal sterile field boundary. Prior art approaches have used separate wrapping or draping on the C-arm itself. This creates problems since parts of the C-arm still move in and out of the sterile field as it is rotated. Vertical drapes of the type described above are also in common use with C-arms. Here parts of the C-arm may pass under or over the drape or through a hole in the drape. However, this has the problem of violating the sterile field. Also, prior art drapes usually have a fluid reservoir centrally located to receive and trap bodily fluids. This is in the way of entry of the C-arm. Forcing part of the C-arm through a hole in the drape or under the drape has the tendency to spill the contents of these reservoirs causing a mess and exposing personnel to biofluids. It would be advantageous to have a clear plastic isolation drape that allowed entry of a C-arm without violating the sterile field and without interfering with fluid reservoirs.

### SUMMARY OF THE INVENTION

The present invention relates to a sterile isolation drape that can be used to create a vertical boundary to the sterile field during hip fracture and similar surgeries. The invention includes a cutout region or opening in the drape that interfaces with an elongated, closed plastic tunnel. This closed tunnel can receive the lower part of the C-arm and allow it to extend into position with respect to the patient without entering the sterile field. A fluid reservoir can be located laterally on one or both sides of the tunnel region to allow trapping of body fluids such as blood without interfering with the entry of the C-arm. The drape can be weighted on the bottom so that the vertical portion remains substantially vertical during entry and exit of the C-arm. The weights can be fixed or removable. The drape can also have optional utility pouches in convenient locations.

### DESCRIPTION OF THE FIGURES

Attention is now directed to several figures the illustrate features of the present invention:

FIG. 1 shows a flat view of a prior art drape.

FIG. 2 shows a flat view of an embodiment of the present invention.

FIGS. 3A, 3B, 3C and 3D show a side schematic view of the drape of the present invention with a C-arm inserted and positioned at various angles.

Several drawings and illustrations have been presented to aid in understanding the present invention. The scope of the present invention is not limited to what is shown in the figures.

### DESCRIPTION OF THE PREFERRED EMBODIMENTS

The present invention relates to a sterile vertical boundary isolation drape that allows easy entry of a C-arm without violation of the sterile field.

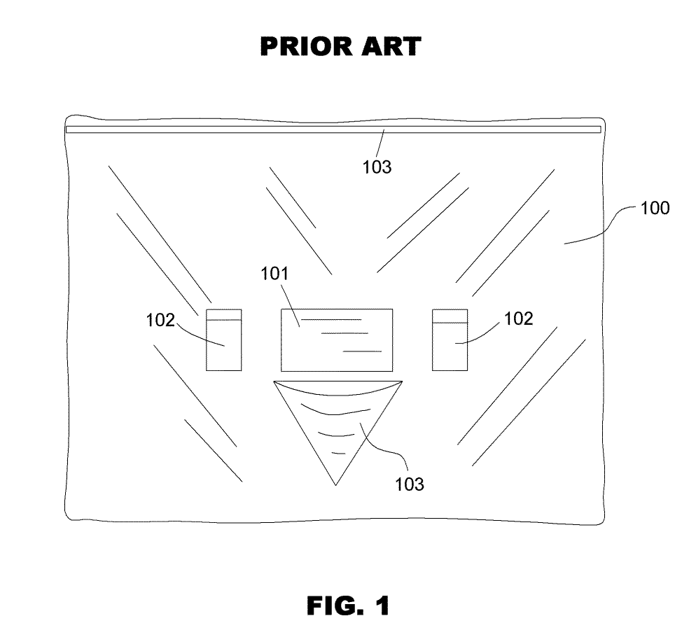

FIG. 1 shows a prior art isolation drape. The drape 100 has a covered incise film 101 and a body fluid reservoir 103 along with a pair of utility pouches 102. It is also is equipped with paper-covered sticky tape 104 along the top for hanging. It can be seen that there is no place to insert a C-arm except through the incise film or under the drape.

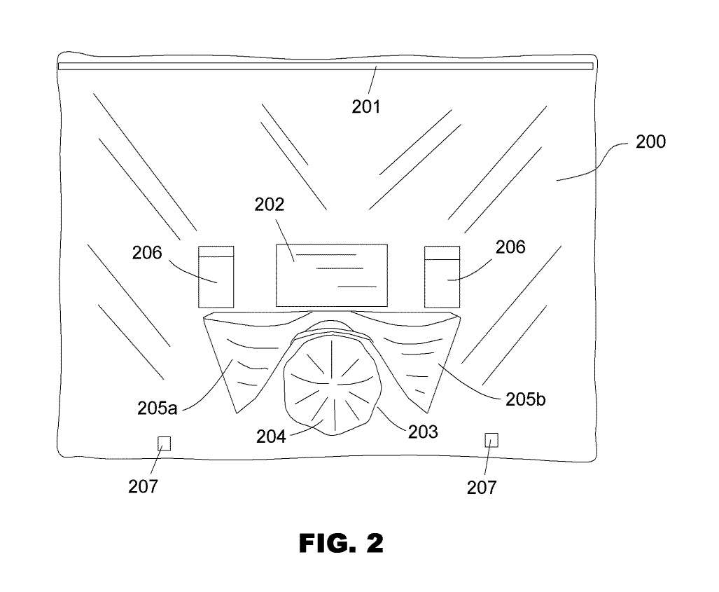

FIG. 2 shows an embodiment of the present invention. The drape has a substantially rectangular flat body 200 made from a flexible, translucent plastic material. The drape body 200 has paper-covered sticky-tape 201 at locations on its edges or periphery for easy hanging and removal. The drape body 200 can include an incise film 202 like prior art drapes. The drape body of the present invention however, has an opening 203 below the incise film 202 adapted to receive a C-arm. This opening 203 can be circular or any other shape. A typical size of the opening is several feet in diameter to accommodate a C-arm. The opening 203 interfaces with an elongated plastic tunnel 204 that is closed at its far or distal end and open at its near or proximal end. The tunnel is also made from the same, or similar, flexible, translucent plastic material. The near end of the tunnel 204 is open and is sealed around the opening 203 in the drape to preserve the sterile field when a C-arm is inserted into the opening in the drape and into the elongated tunnel member. The combination of the opening in the drape and the tunnel member forms a single functioning unit.

Optionally, the drape body can include one or more fluid receiving reservoirs 205a, 205b offset laterally with respect to the opening 203 in the drape. Additionally, the drape body can be equipped with several optional weights 207 near the lower edge chosen to hold the drape body 200 in a vertical configuration when a C-arm is inserted into the elongated tunnel 204. The weights can be fixed or removable. Finally, the drape can be equipped with several service pouches 206 on the sides of the incise film 202 similar to those of the prior art device shown in FIG. 1.

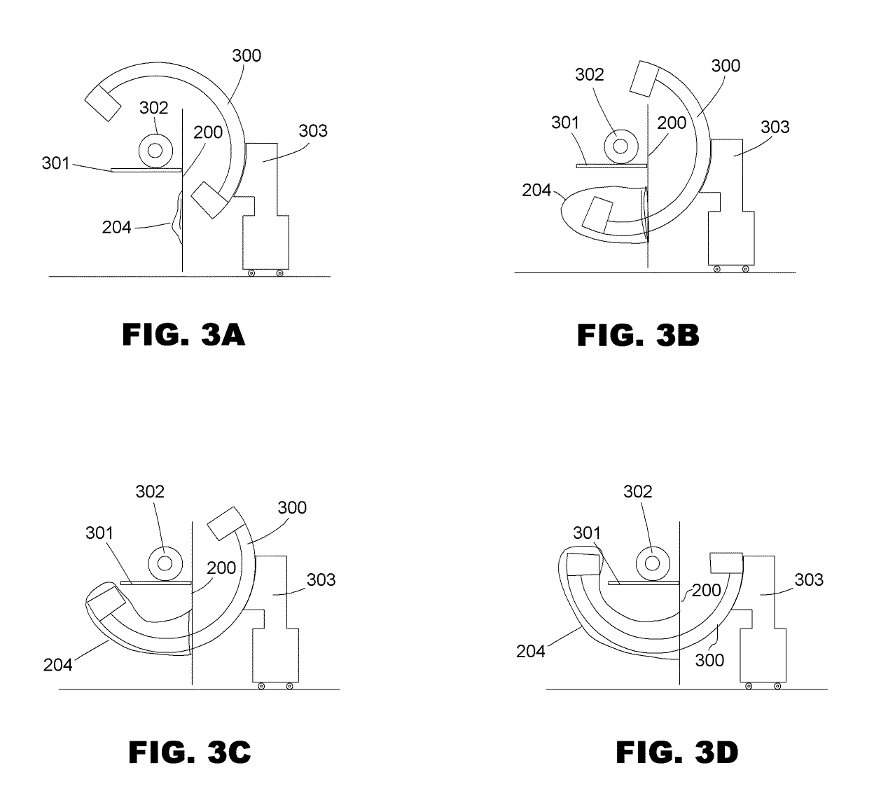

FIGS. 3A-3D show a side schematic view of a C-arm 300 inserted into the elongated tunnel 204 at various angles. The C-arm can be rotated to any angle or tilt during the surgical procedure without violating the sterile field. FIG. 3A shows the C-arm 300 and its associated equipment at an angle where the lower end is not inserted into the tunnel 204. The drape 200 hangs vertically to form a boundary between the table 301 and part of the patient in the sterile field and the non-sterile area where the C-arm support 303 is located. Here, in real surgery, at this angle, the top of the C-arm would also be separately draped since it extends into the sterile field. FIG. 3B shows the C-arm 300 rotated to an angle where the lower part enters the elongated tunnel 204 in the drape 200. The sterile field is maintained. FIG. 3C shows an even more extreme angle where the C-arm 300 penetrates almost the entire length of the tunnel 204, while FIG. 3D shows the C-arm at a maximum angle. In each case, the sterile field is maintained.

When not in use, the elongated, flexible tunnel 204 can be simply folded back flush with the drape body as shown in FIG. 3A. When it is desired to insert the C-arm, the tunnel extends inward accepting the lower part of the C-arm at any angle as shown in FIGS. 3B, 3C and 3D.

The entire drape with the tunnel can be supplied in a folded configuration in a sealed sterile container. The weights can optionally be supplied separately, or the drape, as supplied, can have the weights attached. Upon opening the sterile container, the surgical suite staff can easily hang the drape by attaching its upper edge to any convenient structure by pealing the paper cover from the sticky tape 201 (FIG. 2) and mounting the drape using the sticky tape or otherwise.

While clear, thin plastic such as Mylar™ is the preferred material, any translucent, flexible material may be utilized and is within the scope of the present invention.

Several descriptions and illustrations have been presented to aid in understanding the present invention. One with skill in the art will realize that numerous changes and variations may be made without departing from the spirit of the invention. Each of these changes and variations are within the scope of the present invention.

I claim:

1. A sterile isolation drape adapted to maintain a sterile field in surgery comprising:
a substantially rectangular drape body made from a flexible, translucent material, the drape body having sticky-tape at locations on its periphery for hanging;
the drape body having an opening adapted to receive a C-arm, the opening interfacing with an elongated tunnel member closed at a distal end and open at a proximal end also made from a flexible, translucent plastic material; wherein the open proximal end of the tunnel member is sealed around the opening in the drape to preserve the sterile field when a C-arm is inserted through the opening in the drape and into the elongated tunnel member;
the drape body having at least one fluid receiving reservoir offset laterally with respect to said opening;
the drape body having a plurality of weights in proximity to a lower edge adapted to hold the drape in a vertical configuration when a C-arm is inserted into said elongated tunnel member.

2. The isolation drape of claim 1 wherein the weights are removable.

3. The isolation drape of claim 1 wherein there are two fluid receiving reservoirs offset laterally left and right from a vertical centerline.

4. The isolation drape claim 1 wherein the drape and tunnel member are made from clear flexible plastic.

5. The isolation drape of claim 1 further comprising an incise film located above said opening.

6. A sterile isolation drape adapted to maintain a sterile field in surgery comprising:
a substantially rectangular drape body made from a flexible, translucent material, the drape body having sticky-tape at locations on its periphery for hanging;
the drape body having an opening adapted to receive a C-arm, the opening interfacing with an elongated tunnel member closed at a distal end and open at a proximal end also made from a flexible, translucent plastic material, wherein the open proximal end of the tunnel member is sealed around the opening in the drape to preserve the sterile field when a C-arm is inserted through the opening in the drape and into the elongated tunnel member;
the drape body having at least one fluid receiving reservoir offset laterally with respect to said opening.

7. The isolation drape of claim 1 wherein the drape body includes a at least one weight in proximity to a lower edge adapted to hold the drape in a vertical configuration when a C-arm is inserted into said elongated tunnel member.

8. The isolation drape of claim 7 wherein said weight is removable.

9. Th isolation drape of claim 7 wherein there are two weights.

10. The isolation drape of claim 6 wherein there are two fluid receiving reservoirs offset laterally left and right from said opening.

11. The isolation drape claim 6 wherein the drape and tunnel member are made from clear flexible plastic.

12. The isolation drape of claim 6 further comprising an incise film located above said opening.

13. A method of maintaining a sterile field when using a C-arm with a vertical isolation drape comprising:
providing a substantially rectangular drape body made from a flexible, translucent material, the drape body having sticky-tape at locations on its periphery for hanging wherein, the drape body includes an opening adapted to receive a C-arm, the opening interfacing with an elongated tunnel member closed at a distal end and open at a proximal end also made from said flexible, translucent plastic material, wherein the open proximal end of the tunnel member is sealed around the opening in the drape to preserve the sterile field when a C-arm is inserted through the opening in the drape and into the elongated tunnel member;
providing at least one fluid receiving reservoir offset laterally with respect to said opening on said drape body.

14. The method of claim 13 wherein the drape body includes a plurality of weights in proximity to a lower edge adapted to hold the drape in a vertical configuration when a C-arm is inserted into said elongated tunnel member.

15. The method of claim 14 wherein said weights are removable.

16. The method of claim 13 further comprising a second fluid receiving reservoir.

17. The method of claim 13 wherein the drape and tunnel member are made from clear flexible plastic.

18. The method of claim 13 further comprising an incise film located above said opening.
